(19) United States
(12) Patent Application Publication
(10) Pub. No.: US 2022/0016473 A1
(43) Pub. Date: Jan. 20, 2022

(54) Rehabilitation System

(71) Applicant: Vadim Alexander Gordin, Brooklyn, NY (US)

(72) Inventor: Vadim Alexander Gordin, Brooklyn, NY (US)

(21) Appl. No.: 17/067,625

(22) Filed: Oct. 9, 2020

Related U.S. Application Data
(60) Provisional application No. 62/912,639, filed on Oct. 9, 2019.

(51) Int. Cl. A63B 22/00; A63B 21/00; A61F 4/00; A63B 21/012

(52) U.S. Cl. CPC A63B 22/0005; A63B 21/4049; A63B 2022/0094; A63B 21/0125; A61F 4/00

(57) ABSTRACT

Systems are disclosed for providing rehabilitation for hand and arm impairment after a stroke or other neurodegenerative condition.

### BACKGROUND/FIELD

This application claims priority to provisional application No. 62/912,639 filed on Oct. 9, 2019 which is incorporated by reference herein in its entirety.

The invention relates to hardware and software systems for physical rehabilitation and exercise after stroke or other neuro-degenerative condition.

### SUMMARY

According to certain embodiments of the present disclosure, a rehabilitation system comprises a physical controller.

According to certain embodiments of the present disclosure, a rehabilitation system comprises adaptive gameplay responsive to a user's aptitudes.

### BRIEF DESCRIPTION OF THE FIGURES

In the figures, which are not necessarily drawn to scale, like numerals describe substantially similar components throughout the several views. The drawings illustrate generally, by way of example, but not by way of limitation, various embodiments discussed in the claims of the present document.

FIG. 1 shows a perspective view of a rehabilitation system;

FIG. 2 shows a perspective view of a partially disassembled rehabilitation system;

FIG. 3 shows a perspective view of roller;

FIG. 4 shows a side view of a roller;

FIG. 5 shows a perspective view of a further roller

FIG. 6 shows a side view of a further roller;

FIG. 7 shows a perspective view of an embodiment of a grip;

FIG. 7a shows a side view of an embodiment of a grip;

FIG. 8 shows a perspective view of a handle;

FIG. 9 shows a perspective view of a further embodiment of a rehabilitation system;

FIG. 10 shows a front view of a rehabilitation system.

FIG. 11 shows a perspective view of a rehabilitation system;

FIG. 12 shows a bottom view of a rehabilitation system;

FIG. 13 shows a perspective view of a rehabilitation system;

FIG. 14 shows a perspective view of a rehabilitation system;

FIG. 15 shows a perspective view of a grasping token;

FIG. 16 shows a perspective view of a compressible button;

FIG. 17 shows a perspective view of a rotable insert

FIG. 18 shows a software and hardware flow chart for a rehabilitation system.

FIG. 19 shows an example screen of gameplay within a rehabilitation system.

### DETAILED DESCRIPTION OF THE FIGURES

Various non-limiting embodiments of the present disclosure will now be described to provide an overall understanding of the principles of the structure, function, and use of the proficiency tracking systems and processes disclosed herein. One or more examples of these non-limiting embodiments are illustrated in the accompanying drawings. Those of ordinary skill in the art will understand that systems and methods specifically described herein and illustrated in the accompanying drawings are non-limiting embodiments. The features illustrated or described in connection with one non-limiting embodiment may be combined with the features of other non-limiting embodiments. Such modifications and variations are intended to be included within the scope of the present disclosure.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment,” or “in an embodiment” in places throughout the specification are not necessarily all referring to the same embodiment. Furthermore, the particular features, structures or characteristics may be combined in any suitable manner in one or more embodiments.

Described herein are example embodiments of systems and methods for providing rehabilitation exercises and games to persons having hand and arm impairments.

The examples discussed herein are examples only and are provided to assist in the explanation of the apparatuses, devices, systems and methods described herein. None of the features or components shown in the drawings or discussed below should be taken as mandatory for any specific implementation of any of these the apparatuses, devices, systems or methods unless specifically designated as mandatory. For ease of reading and clarity, certain components, modules, or methods may be described solely in connection with a specific figure. Any failure to specifically describe a combination or sub-combination of components should not be understood as an indication that any combination or sub-combination is not possible. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

Specific sizes and scales are here provided solely as non-limiting examples of implementation(s) of the present invention. The substance of the present invention is an arrangement of functional elements as described in the claims that make it easier to conduct rehabilitation after stroke than with any systems described in the prior art.

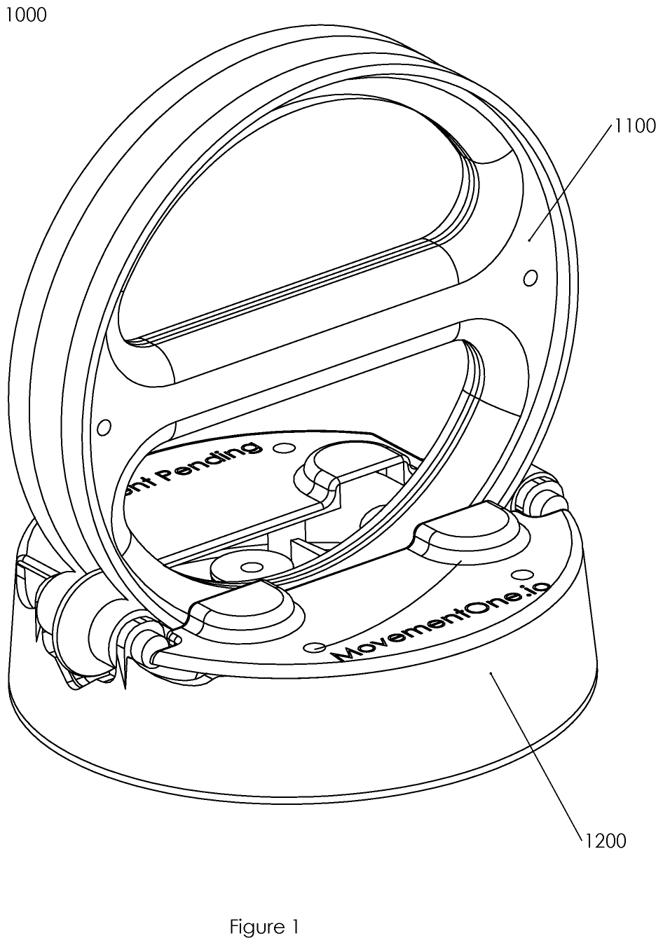

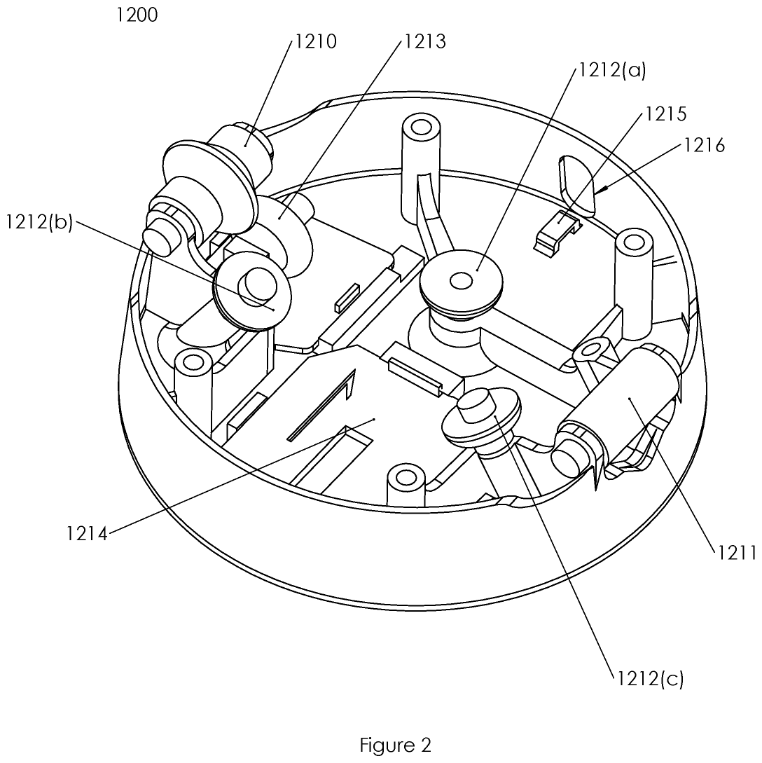

Referring to FIG. 1, a rehabilitation tool 1000 includes a rotating grip 1100 which captively rotates within a base body 1200. An embodiment of a base in shown in FIG. 2 with the grip 1100 and lids removed. There is a plurality of rollers 1212(a-c) rotably coupled to the base body 1200 in tangential relation with an outer bearing surface of the grip. The rollers 1212(a-c) provide lateral constraint to the grip. There are two end rollers 1210 and 1211 rotably coupled to the body upon which the grip 1100 rests and can rotate. There is an optical mouse sensor 1214 disposed above a window in a bottom face so that the optical mouse sensor can see the surface upon which the device is resting. There is a rotary encoder 1213 which rolls with the grip via frictional engagement, thereby capturing and encoding its movement. The optical mouse sensor and rotatory encoder are digitally connected to a processor which translates the recorded movements into mouse movements understandable by a desktop computer.

There is a strain relief 1215 to which an output wire can be connected via an aperture 1216.

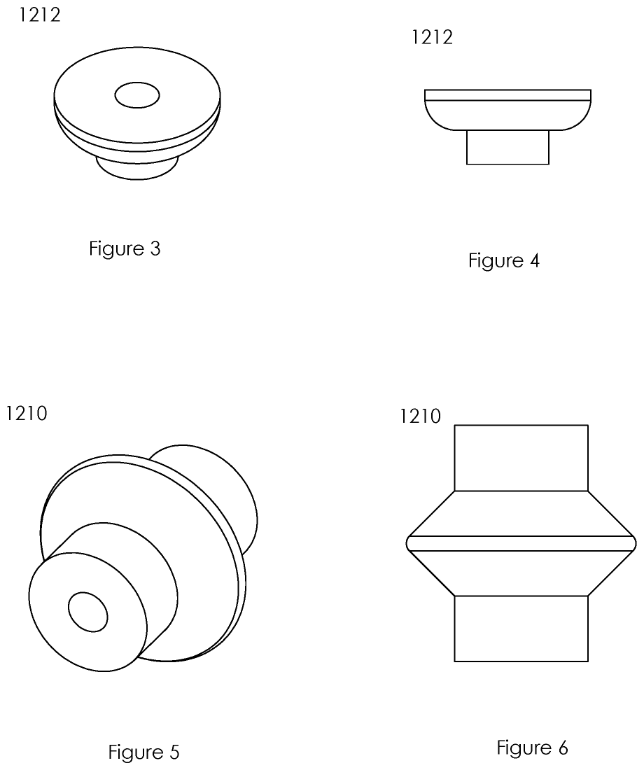

Referring now to FIGS. 3 and 4 together, roller 1212 is a body having a round cross-section with a cylindrical bottom portion and an arcuate top portion and a cylindrical aperture running therethrough . Upon assembly as shown in the previous figures, the cylindrical portion is placed in a tangential relation to the outer face of the grip 1100, which the arcuate portion provides the downward seating force upon the grip.

Referring now to FIGS. 5 and 6 together, an end roller 1210 is shown having cylindrical portions upon opposing ends thereof with opposing joined frustoconical sections therebetween. The frustoconical sections are sized and shaped to roll within a complementary channel in the bearing surface of grip 1100 which the flat bearing surface of grip 1100 rests upon the cylindrical portions.

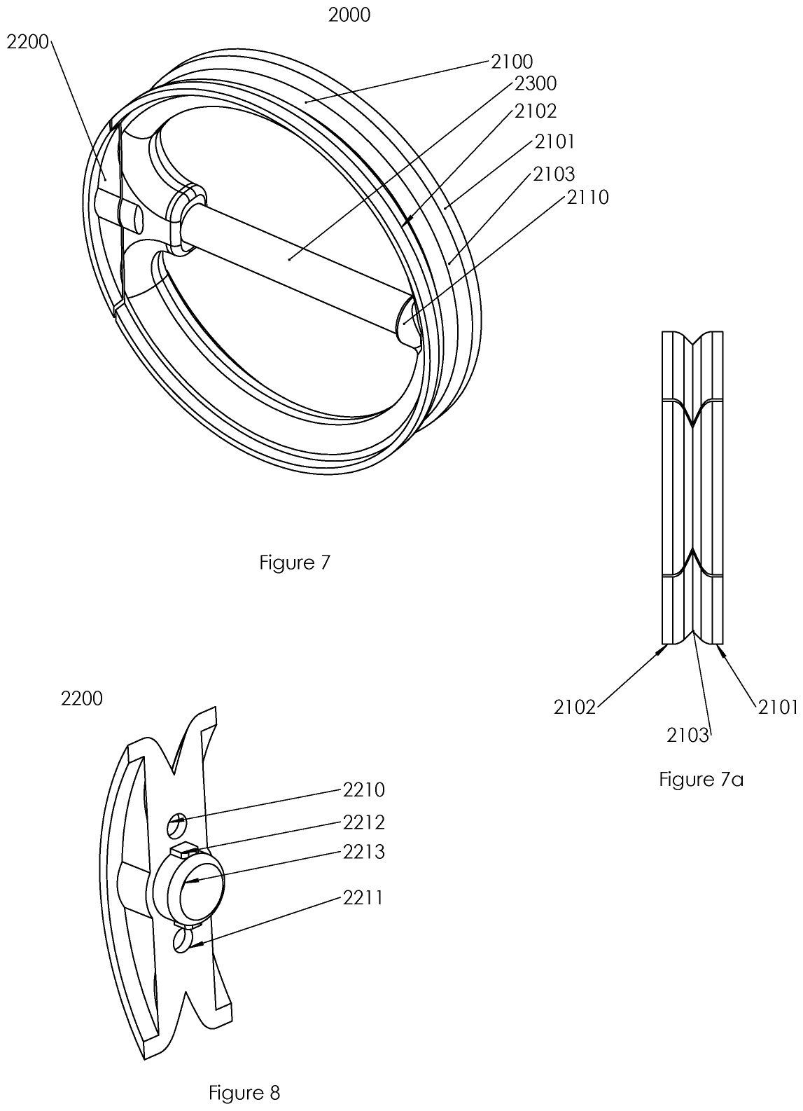

Referring now to FIGS. 7 and 7a together, a further embodiment of a rotating grip 2000 is shown, with the grip having an outer body 2100, a handle 2200 and a pin 2300. Handle 2100 generally approximates a ring with a channel 2103 and an outer bearing surface 2102 and 2101.

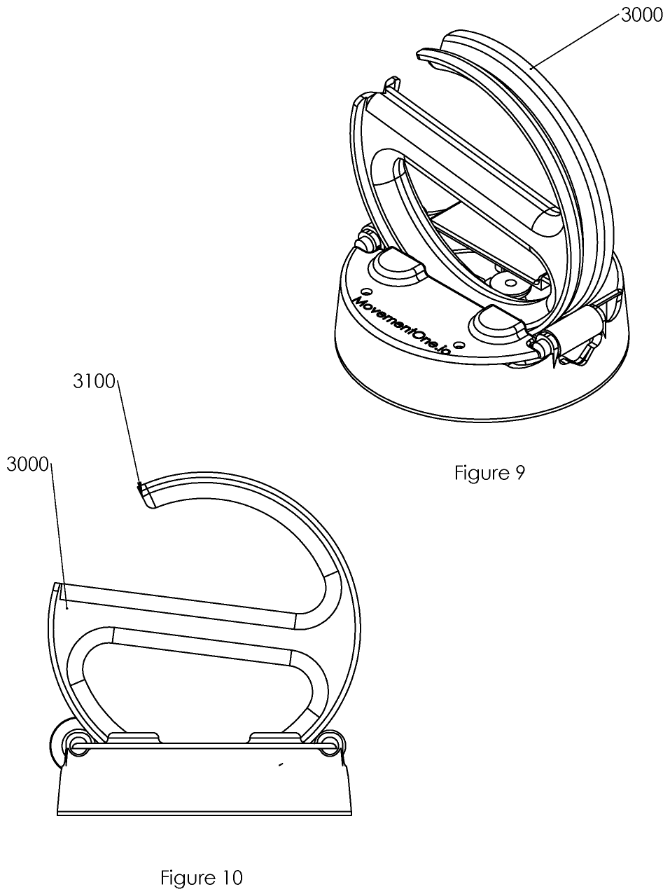

Referring now to FIG. 8 a handle 2200 is shown having a grasping portion at a first end thereof and a protrustion 2213 at a second end thereof. Protrusion 2213 is a generally tubular body sized and shaped to permanently retain the pin 2300 therewithin by means of friction or adhesive. There are alignment tabs 2212 sized and shaped to rest within complementary recesses within the handle 2200 to prevent rotation about the long axis of pin 2300. There are apertures 2210 and 2211 sized and shaped to retain magnets therewithin which align with complementary metal slugs in grip 2100. The pin and handle are capable of being slide in and out of body 2100 by a user thereby approximating an open ring when the pin and handle are removed and and a bisected ring (as shown in FIG. 7) when the pin and handle are inserted. Upon insertion, the pin and handle are urged into place and retained in place by the force of the magnets upon the metal slugs.

According to further embodiment of rotating grip 3000 shown in FIGS. 8 and 9, the rotating grip 3000 has a cutout 3100 in the outer perimeter thereof. The spacing of the cutout is smaller than the angular spacing of the rollers 1212(a-c), such that the grip remains engaged and sufficiently constrained to rotate steadily and smoothly. The purpose of the cutout 3100 is to allow a stroke patient whose hand is stuck in involuntary flexion to place their hand upon the grip.

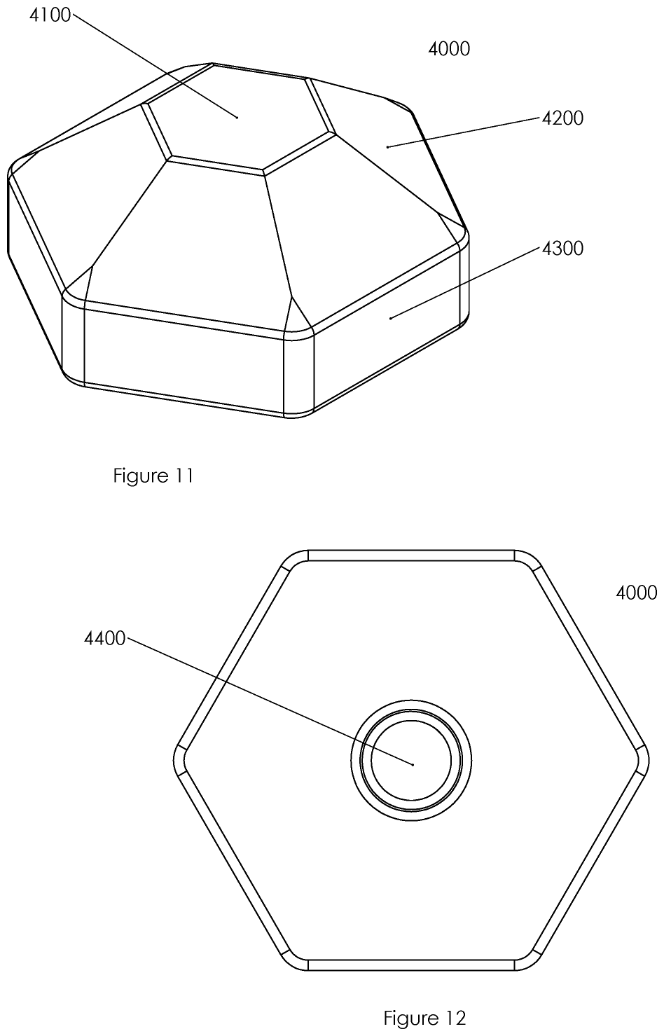

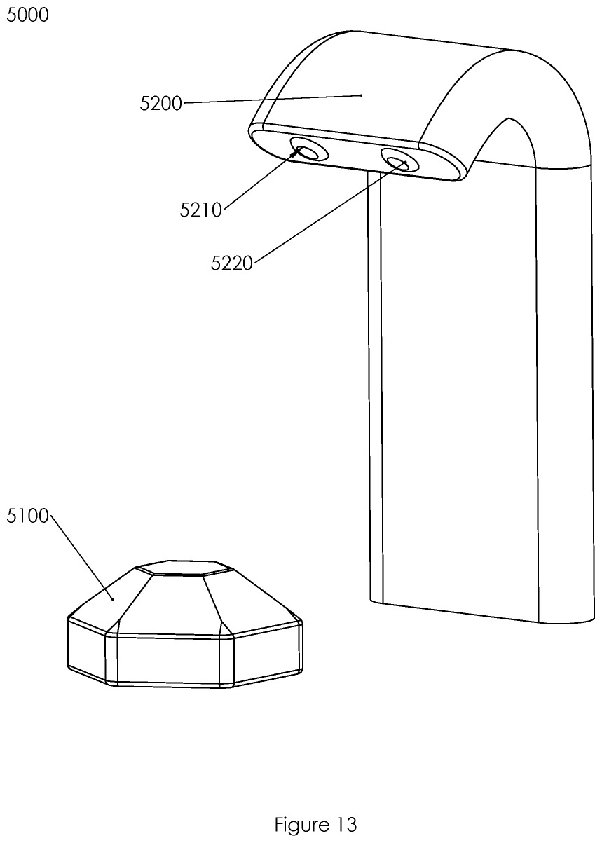

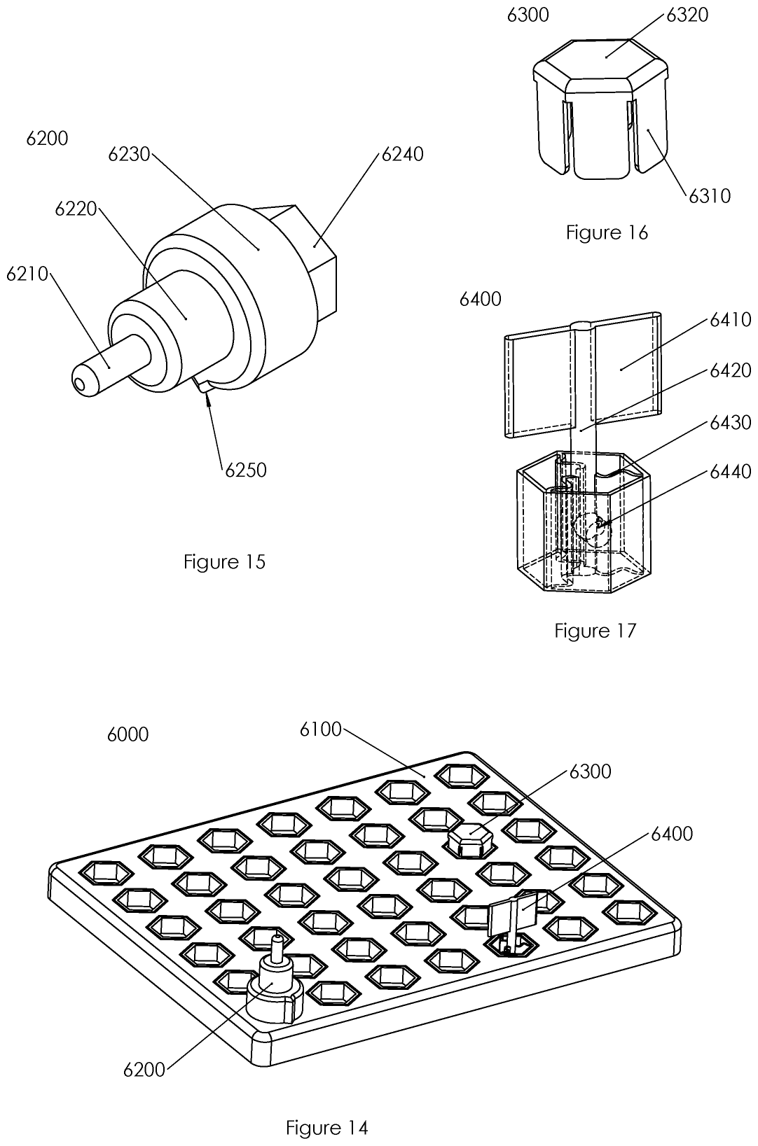

Referring now to FIGS. 11 and 12, a training puck 4000 is shown, with the puck having a plurality of angled faceted surfaces 4200 meeting a top face 4100 thereof, and a plurality of vertical faceted surfaces 4300 which connect the angled faceted surfaces 4200 to a bottom surface. The bottom surface has a pushbutton 4400 disposed in the face thereof. There is a Bluetooth transceiver, processor, and battery within the device. There are capacitive sensors upon each face, as well as an accelerometer and gyroscope within the device. The button activates the Bluetooth transceiver and initiated a connection scheme. The Bluetooth transceiver mimics a keyboard and transmits ASCII codes that correspond to the reading from the capacitive sensors, accelerometer, and gyroscope. For instance, there may be a plurality of letters mapped to each of the respective faces facing downward as detected by the gyroscope. Similarly, there may be letters mapped to the capacitive sensors detecting the presence of a user's grip nearby. Finally, there may be letters mapped to acceleration in various directions as detected by the accelerometer.

According to further embodiments of the present disclosure, the accelerometer or gyroscope is mapped to XY coordinates and communicated to the computer as movements of a mouse. By mimicking conventional input devices such as keyboards and mice, the device can communicated its position in space to a computer without the need for proprietary device drivers.

According to a further embodiment of the present disclosure, a puck 5100 is shown. Puck 5100 is geometrically similar to puck 4000, but without any internal electronics. Instead, puck 5100 has faces that are optically recognizable by a camera 5210 on an armature 5200 by means of their shape or another visual marking. The scene as recorded by camera 5210 is interpreted by a feature recognition machine vision system which determines the orientation and position of the puck 4000. A corresponding game environment is then projected downward onto the puck 4000 and its surrounding environment by a projector 5220.

Referring now to FIGS. 15, 16, and 17, a pegboard system 6000 is shown. A pegboard base 6100 has a plurality of hexagonal (or other faceted shape) apertures disposed in at least one face thereof, with each aperture being surrounded by light-pipe driven by at least one LED. Each aperture has at least one sensor for detecting the present of an element therein. The sensor may be for instance a interruptible line of sight sensor, a capacitive sensor, or a mechanical switch. There are a plurality of tokens usable with the pegboard including a grasping token 6200, a pushbutton token 6300, and a twisting token 6400. According to certain embodiments of the present disclosure, the graspable token 6200 is comprised of conductive material, including for instance graphite doped plastic, such that the measurement recorded by an inductive sensor in a given aperture upon the pegboard base records a distinct reading if a use grasps a small diameter portion 6210, a medium diameter portion 6220, or a large diameter portion 6230. According to certain embodiments of the present disclosure, the base 6240 of the graspable token 6200 is a polygon complementary to the shape of the apertures disposed in the pegboard base. One of the faces of the base 6240 may be of greater conductance than the others, including for instance by the presence of a portion of metal, such that an inductive sensor disposed within an aperture of the pegboard base is able to discern the rotational position of the gaspable token within the aperture.

Referring now to FIG. 16, a compressible button 6300 is shown, wherein the button is comprised of a flexible material, including for instance a silicone or TPU, with the compressible button having a solid top 6320 and a plurality of compressible tines 6310. When inserted into an aperture of the pegboard base having a capacitive or inductive sensor therewithin, the output of the sensor can be used to approximate the force with which a user pushes down onto the top 6320 of the compressible button.

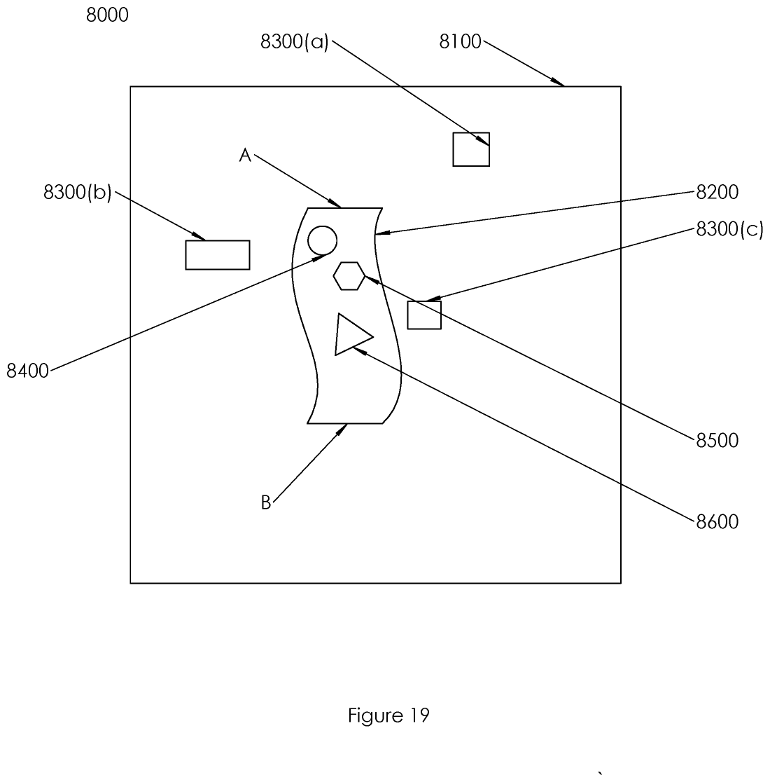

Referring now to FIG. 17, a rotable insert 6400 has a paddle grip 6410 at a first end thereof, extending down into a shaft 6420 therebelow. The rotable insert is comprised of a complaint material, including for instance silicone or TPU. The bottom portion of the shaft terminates into a group of flexible veins 6430 with radiate outward to an outer shell which is sized and shaped to be inserted into an aperture of a pegboard base. There is a magnet or metal slug 6440 coupled to the bottom portion of shaft 6420. In use, when the rotable insert 6400 is placed into an aperture having a inductive or capacitive sensor therewithin, the readout of the sensor can approximate the rotational position of the paddle grip. For instance, if a user grasps the paddle grip and twists it, thereby radially displacing the slug 6440, the readout of the capacitive or inductive sensor responds accordingly, providing an approximate measurement of both the radial displacement of the paddle grip and the force applied by the user.

In use, the various states of the pegboard system 6000 may be mapped to a keyboard list, such that the relative position of a piece in the pegboard is communicated to a computer as an asci character. In the same vein, the pegboard system may receive input from a computer via encoding via a headphone jack. In such a way, a user may visit a gaming website in order to use the pegboard. The gaming website plays a sound which communicates to the pegboard by way of a modulation scheme such as amplitude modulation, frequency modulation, phase key shifting, or other encoding scheme a specific LED to illuminate about a specific aperture. The web site then displays a countdown timer the counts until a user places the appropriate piece into the appropriate aperture. Once this is done, the pegboard communicates an asci code to a website that corresponds to the aperture engaged and the website responds accordingly, including for instance by restarting the timer and illuminating/highlighting a new aperture. According to certain embodiments of the present disclosure, different tokens contain different machine readable elements by which an aperture may identify those tokens with appropriate readers, including for instance RFID tokens or bar-code tokens.

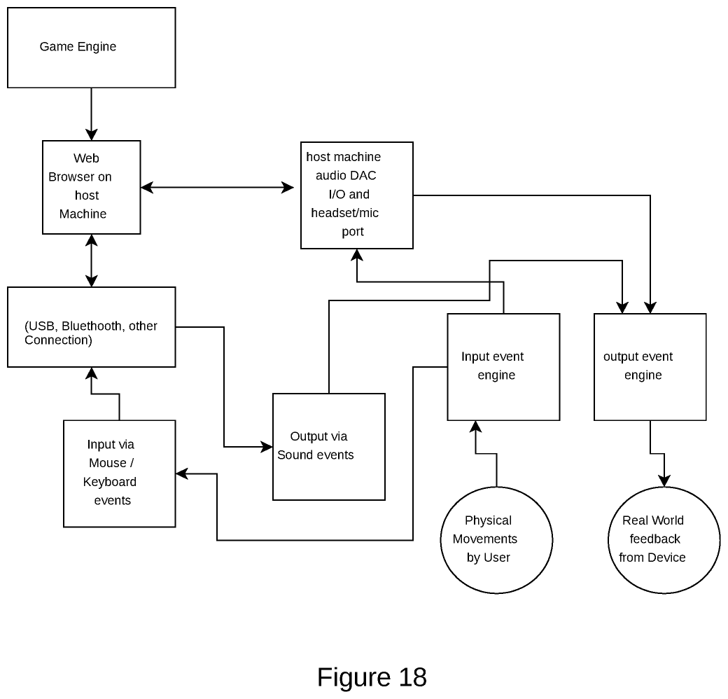

With reference to FIG. 18, according to certain embodiments of the present disclosure, the preceding physical devices are electronically connected to computers so as to mimic conventional input devices such as mice and keyboards. For instance, in the case of rehabilitation tool 1000, the system mimics a computer mouse, communicating its position on a tabletop as mouse XY coordinates and the rotary position of the grip 1100 as a horizontal or vertical scroll. According to further embodiments of the present disclosure a training puck 4000 can communicate its orientation and position in terms of one of pitch, yaw, or accelerations from the internal accelerometer or gyroscope as one of the axis of output of a mouse. Similarly, each of the faces of the puck 4000 may be associated with a different letter which is communicated to the computer as a keyboard output. In the instance of a pegboard 6000, the device may communicate bi-directionally. Specifically, the device may take input by being connected to the headphone jack of a computer or as a USB or Bluetooth soundcard in addition to as a mouse or keyboard. In such a configuration, a game on a website may communicate which LEDs should be illuminated with data encoded into sounds through modulation. Conversly, there maybe letters or asci codes associated with each aperture, which are transmitted to the computer to signify why that aperture has been engaged by a user and similarly a range of outputs of the capacitive sensor for a given aperture. For instance, the letters a, b, c, d, e may all refer to a single aperture and represent the state of the capacitive sensor, thereby allowing the choice of letter to be representative of the amount of downward force being applied by a user to a pushbutton 6300, the amount of rotational force being applied to a twisting token 6400, or the relative rotational position of a grasping token 6200.

Referring now to FIG. 18, one method of configuring the software and hardware elements of the computer interaction interface of the preceding sections is shown. A game engine is provided, including for instance game engines and environments such as Phaser, Contruct, Unity, Flash or other development platforms and environments capable of displaying a video game capable of receiving user inputs to physical devices. The game engine operates within a web browser which runs on a host machine, including for instance a desktop computer, laptop computer, phone, or tablet operated by a user. The web browser receives user input by means of electronic communication via USB, Bluetooth, or other connection scheme to a physical controller which is manipulated by a user. The physical movements of the user are converted by an input event engine into traditional keyboard or mouse movements. The benefit of such an approach is that peripherals such as those described can then be used to operate a web-browser based game without requiring any proprietary driver software on a user's computer.

In the same vein (but opposite data direction of data travel), outputs from a game engine may be communicated to a peripheral such as the controllers previously described by means of either the onboard sound card of a computer or the peripheral mimicking a USB or Bluetooth sound card when connected. In a first embodiment of such a scheme, a peripheral such as pegboard 6000 may have a wire for plugging into a headphone jack. This wire would then be in electronic communication with a microprocessor configured to demodulate signals encoded in the audio played by a website. In turn, the website would then transmit modulated signals via its sound output which communicate to the pegboard which if any LEDs within the pegboard should be illuminated at any given time. In a second embodiment of such a scheme, a microprocessor within the peripheral is configured to act as a Bluetooth or USB sound card, so that when modulated signals are received from the website as encoded sounds, the microprocessor is capable of demodulating these into data usable by the peripheral. According to further embodiments of the present disclosure, the audio communication scheme described in this section is used to drive haptic feedback such as eccentric vibration motors, or physical actuators such as servos or linear actuators, or electric motors which affect the physical state of the peripheral.

According to further embodiments of the present disclosure, the gameplay within the system is continuously modified to adapt to the cognitive abilities of a user, physical abilities of a user, and noise rejection abilities of a user. In terms of physical abilities, the system has several classes of aptitudes, including for instance speed of movement, distance of movement, steadiness of movement, decision time, accuracy, endurance for sustained movement, endurance for repetitive movement, and range of motion. In terms of cognitive abilities, the system has several classes of aptitudes, including for instance sustained attention, selected attention, divided attention, multiple simultaneous attention, short term working memory, long term working memory, category formation, and pattern recognition. In terms of noise rejection abilities of a user, the system has several classes of aptitudes, including for instance extraneous visual data like quantity of extraneous objects, colors of extraneous objects, stillness or motion of extraneous objects, position of extraneous objects, and the size of extraneous objects. Further examples of noise include acoustic data such as different wavelenths of sounds different rhythmic patterns, constant noise, random noise, noise associated with game events, music, and ambient/steady noise.

The system constantly tracks how user responds to the various challenges and stimulae described above in order to continuously manipulate gameplay to maintain an experience that is both challenging for a user and drives progress in rehabilitation.

The system for modifying gameplay as described in the preceding sections will now be described with specific reference to 4 different types of games, specifically a Tetris clone, a ‘target practice’ style game, a ‘moving road’ style game, and a turn based game such as chess, checkers or scrabble.

In the case of a tetris clone, a controller such a rehabilitation tool 1000 is connected via USB to a computer and configured to communicate its position on a tabletop to the computer as mouse XY coordinates and the rotational position of grip 1100 as vertical scroll position. A game engine is disposed within a website. When a user visits the game, the game engine initials displays a series of calibration screens asking the user test their range of motion on a given day. The user is asked to move their arm up as far as possible and then press a mouse button or spacebar, down as far as possible followed by spacebar, left as far as possible followed by spacebar, to pronate their hand as far as possible followed by spacebar, and to supinate their hand as far as possible followed by spacebar. The website records these movements (which may extend beyond the screen space of the website using functionality such as Pointer Lock within HTMLS). The calibration data is both recorded as one measure of historical range of motion for a user and also used to scale further interactions with the game. Once gameplay begins, a user is able to move pieces left/right by manipulating the controller left/right, drop pieces down by nudging the controller downward, and rotate the pieces by rotating the grip. The game engine keeps track of speed and range of motion by monitoring movements during gameplay. Similarly, the system keeps track of how long from when a block appears until when a user orients it into its final position to as measures of motor planning and decision/processing time. As the game progresses the engine first establishes a baseline for the user's performance and then begins injecting noise and challenges and tracking how well the user's performance changes. For instance, the system can change how quickly the next block is revealed as well as speed of falling to challenge reaction/processing time, it can initially start in black and white and then slowly introduce colored tiles to challenge ability to process visual noise, and it can require different levels of movement of the controller in order to correspond to translations of the tetris pieces on-screen. The measurement and manipulation of these elements happens dynamically and in a manner that is transparent to the user.

In the case of a “target practice” game such as “duck hunt” for NES, a sighting cross-hair is provided on-screen which is manipulated by means of moving the controller 1000 on a tabletop. Rotation of the grip may also be included to draw back a bow/arrow or cock a gun within the gameplay analogy. The game engine could then deploy targets in accordance with a user's abilities, for instance varying the number of targets on the screen, their speed, where they appear relative to the user's cross-hairs, the size of their target areas, the number, quantity, and type of extraneous elements on the screen and where they are relative to the user's crosshairs, etc. in order to both measure a user's abilities and challenge them to improve.

Referring now to FIG. 19 a ‘moving road’ style game is shown wherein a road 8200 is shown within a game area 8100. The road has a head A at a first end thereof and a tail B at a second end thereof. The gameplay is such that new roadway is added to the head while old roadway is destroyed at the tail. As new roadway is added to the head, the game engine can selectively introduce turns, changes in roadway width, on alter the speed of both roadway creation and destruction. The object of the game is for a user to manipulate an avatar 8600 safely on the road without falling off by manipulating a physical controller such as 1000. The system may also introduce extraneous objects such as 8300(a-c) as noise, goals/targets such as 8400, and obstacles to be avoided such as 8500. In the course of gameplay, the system tracks both performance of a user in terms of how long they are able to stay alive by keeping the avatar on the road and as close to the midline of the road as possible as well as what sorts of challenges and goals worsen a user's performance. The game engine can vary both the quantity and quality of goals and obstacles as well as the amount of time between when they are introduced and a user must respond to them. Similarly, the game engine can also vary the amount of physical translation of the controller required to produce movements of the avatar on screen. Over the course of both multiple and single gameplay sessions, the game engine collects data on both the user's aptitudes as well as the sorts of challenges that impede their performance. The system then progressively deploys more of those challenges in order to condition the user to perform better.

According to further embodiments of the present disclosure, a turn-based game such as chess, checkers, scrabble, or the like is provided with the user being able to play against either a computer or a remote human player. In the case of a remote human player, the remote player may be a healthy user who interacts with the game using a traditional computer interface such as a mouse of cell-phone touch screen. In the case of the user (local user) who has a physical impairment and is using one of the aforementioned controllers or physical devices, the local user must complete a physical or cognitive challenge in order to take their turn. For instance, in the case of a game of checkers or tic-tac-toe, a healthy remote user may initiate a game with a local user of a controller 1000. A remote user may mark their “X” or “O” directly onto the screen, after which the game engine communicates the result of the remote user's turn to the local user and invites the local user to take his/her turn as well. The local user engaging with the controller 1000 however would need to perform a randomly selected challenge or exercise which goes to one of the aforementioned aptitudes, including for instance a speed or range of motion exercise, a prolonged motion such as tracing a shape to develop accuracy or endurance, or a working memory exercise with flash-cards. Only upon successful completion of the exercise is the local user able to take his or her turn in the game of tic-tac-toe.

According to further embodiments of the present disclosure, player data is collected which tracks how past players' abilities have previously improved upon exposure to various physical and cognitive challenges. The game engine then uses machine learning to analogize the progress of new players to previous players and thereby determine gameplay changes that will result in positive outcomes or improvement in physical ability, cognitive aptitude, or noise rejection.

Unless otherwise indicated, all numbers expressing quantities of components, conditions, and otherwise used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

It is understood that, in light of a reading of the foregoing description, those with ordinary skill in the art will be able to make changes and modifications to the present invention without departing from the spirit or scope of the invention, as defined herein. For example, those skilled in the art may substitute materials supplied by different manufacturers than specified herein without altering the scope of the present invention.

What is claimed is:

1. A system for rehabilitation as shown and described.

2. A method for rehabilitation as shown and described.
